(19) United States
(12) Patent Application Publication
(10) Pub. No.: US 2018/0154123 A1
(43) Pub. Date: Jun. 7, 2018

(54) IMPLANTS AND SYSTEMS FOR ELECTRICALLY ISOLATING ONE OR MORE PULMINARY VEINS

(71) Applicant: Aperiam Medical, Inc., Boise, ID (US)

(72) Inventors: Randell L. Werneth, Boise, ID (US); Alex J. Asconeguy, Murrieta, CA (US); Ricardo David Roman, Chula Vista, CA (US); Kenneth E. Perry, Bainbridge Island, WA (US); Gerald Martin Stobbs, Alpine, CA (US)

(73) Assignee: Aperiam Medical, Inc., Boise, ID (US)

(21) Appl. No.: 15/577,324

(22) PCT Filed: May 27, 2016
(86) PCT No.: PCT/US16/34877

§ 371 Date: Nov. 27, 2017

Related U.S. Application Data
(60) Provisional application No. 62/166,767, filed on May 27, 2015.

(51) Int. Cl. A61M 29/02; A61B 8/12; A61B 18/14; A61F 2/88; A61F 2/90; A61F 2/93; A61F 2/958; A61F 2/966

(52) U.S. Cl. CPC A61M 29/02; A61B 8/12; A61B 18/1492; A61F 2/88; A61B 2018/00375; A61F 2/93; A61F 2/958; A61F 2/966; A61F 2/90

(57) ABSTRACT

Disclosed are devices, systems, and methods for pulmonary vein ostium and pulmonary vein prosthetic implants for creating a region of scar tissue (non-conductive) in or near one or more pulmonary veins and the superior venacava.

### CROSS-REFERENCE TO RELATED APPLICATIONS

This application claims benefit of priority to U.S. Provisional Patent Application Ser. No. 62/166,767, filed May 27, 2015, incorporated by reference herein. This application is also related to U.S. patent application Ser. No. 13/106,343, filed May 12, 2011 and published as U.S. Publication No. 2011/0282343, U.S. patent application Ser. No. 13/457,033, filed Apr. 26, 2012 and published as U.S. Publication No. 2012/0277842, U.S. patent application Ser. No. 13/655,351, filed Oct. 18, 2012 and published as U.S. Publication No. 2013/0109987 and U.S. patent application Ser. No. 13/830,040, filed Mar. 14, 2013 and published as U.S. Publication No. 2013/0204311, all of which are owned by the assignee of the present application and are incorporated by reference herein in their entirety.

### FIELD

The present invention relates generally to implantable prosthetic assemblies. more particularly, the present invention pertains to pulmonary vein ostium and pulmonary vein prosthetic implants for creating a region of scar tissue (non-conductive) in or near one or more pulmonary veins and the superior vena cava.

### BACKGROUND

Atrial fibrillation is a common and dangerous disease. For example, atrial fibrillation patients have a greatly increased risk of stroke mortality. The heart's normal sinus rhythm typically begins in the right atrium and proceeds in a single, orderly wavefront at rates of 60 to 100 beats per minute. Atrial fibrillation disrupts the normal sinus rhythm. During atrial fibrillation multiple wavefronts circulate rapidly and chaotically through the atria, causing them to contract in an uncoordinated and ineffective manner at increased rates from 300 to 600 beats per minute. Symptoms arise from the rapid, irregular pulse as well as the loss of cardiac pump function related to uncoordinated atrial contractions. These uncoordinated contractions also allow blood to pool in the atria and may ultimately lead to thromboembolism and stroke

Current therapies for atrial fibrillation include the use of anti-arrhythmic drugs and anti-coagulation agents. Anti-coagulation agents can reduce the risk of stroke, but often increase the risk of bleeding. Drugs are useful at reducing symptoms, but often include undesirable side effects. These may include pro-arrhythmia, long-term ineffectiveness, and even an increase in mortality, especially of those with impaired particular function. Drug therapy to slow the ventricular response rate, catheter ablation of the atrioventricular node with pacemaker implantation, or modification of the node without pacemaker implantation can be useful to facilitate ventricular rate control, but thromboembolic risk is unchanged, and therefore the patient must remain on anticoagulants with the problems noted above

Other therapies include surgical and catheter ablation of the pulmonary vein. However, these therapies are associated with high complication rates and long procedure times. In addition, administration of surgical and catheter ablation typically requires extensive training in the use and installation of complex technology.

The limitations and drawbacks of these therapies have caused investigators to search for alternative therapies for atrial fibrillation.

### SUMMARY

The present invention discloses implants and systems for creating a region/area of non-conductive tissue in one or more pulmonary veins. The impants and systems relate to pulmonary vein prosthetic implants which are capable of being delivered using endovascular techniques and being implanted at the pulmonary vein ostium and inside the pulmonary vein. The prosthetic implants of the present invention are well-suited for cardiac delivery via a femoral or subclavian artery approach using a delivery catheter.

According to some embodiments, the implants and systems disclosed herein are related to implanted devices that have improved safety profiles and which minimize or reduce collateral damage over current therapies. The implants are configured to create a block atrial tachycardia originating from the pulmonary veins and/or pulmonary vein foci.

One aspect of this disclosure provides a device method of treating an arrhythmia, comprising: selecting a patient, choosing a first implant device for insertion into a first pulmonary vein of the patient, inserting an implant device delivery catheter into the patient, wherein the implant delivery catheter comprises a distal end and a distal portion and the first implant device is positioned in the distal portion, advancing the distal portion of the implant delivery device into the first pulmonary vein, positioning the first implant device relative to the first pulmonary vein near the ostium and or left atrial—pulmonary vein junction; deploying the first implant device within the first pulmonary vein near the ostium and or left atrial—pulmonary vein junction, radially expanding to a diameter larger than the pulmonary vein the first implant device to cause an effect selected from the group consisting of:
stretching of a portion of the pulmonary vein;
stretching of a portion of the pulmonary vein ostium;
stretching of a portion of the left atrial—pulmonary vein junction;
causing micro-tears in a portion of the pulmonary vein;
causing micro-tears in a portion of the pulmonary vein ostium;
causing micro-tears in a portion of the left atrial—pulmonary vein junction;
and combinations thereof; and
   wherein the stretching and micro-tears resulting in the creation of scar tissue formation, the scar tissue at least partially blocking and/or disrupting electrical conduction along the first pulmonary vein, and withdrawing the distal end of the implant delivery device from the first pulmonary vein.

According to some embodiments, the method includes radially expanding to a diameter larger than the pulmonary vein the first implant device to cause an effect selected from the group consisting of:
outwards pressure applied to the pulmonary vein;
outwards pressure applied to the pulmonary vein ostium;
outwards pressure applied to the left atrial—pulmonary vein junction;
and combinations thereof; and
   wherein the outward pressure causing the myocytes to be compressed, reducing their ability to perform normal function.

An additional aspect of the disclosure provides an implant delivery system for disrupting electrical signals traveling along a pulmonary vein by bidirectional stretching of the pulmonary vein wall, the system comprising a delivery catheter having a shaft with a distal end to a proximal end and lumen between the distal and proximal ends, the delivery catheter being configured for insertion into a patient's vascular system to position the distal end proximate a pulmonary vein location, one or more implants having a radial expandable ring or coil configured to deploy within the pulmonary vein, the ring or coil being configured to deliver a force against pulmonary vein wall to provide bidirectional stretching of the pulmonary vein wall in both a radial direction and axial direction to stretch and create micro-tears in the pulmonary vein wall and then to hold open at a diameter slightly larger than normal without recoil, and a delivery device having shaft with a distal end and proximal end, the one or more implants being positioned proximate the distal end of the delivery device, wherein as the implant exit distally from the delivery catheter lumen, exposing the implant within the pulmonary vein for radial deployment.

In some embodiments, the bidirectional stretching creates a two-step biological response in the pulmonary vein wall to promote cellular decoupling, comprising first, an acute response is caused by pressure-induced apoptosis inhibiting chemical exchange of sodium/calcium and disrupting focal electrical wave propagation, and second, a biological response for chronic or long-term isolation/denervation is provided by causing focal endothelial cell proliferation at the implant site.

In some embodiments, the implant is self-expanding.

In some embodiments, the system further comprising one or more balloons to expand the implant.

In some embodiments, the system further comprising an imaging device.

In some embodiments, the system further comprising an ablation device.

In some embodiments, the delivery device is configured to deliver one implant at a time, so that multiple implants may be implanted into a pulmonary vein, or individual implants may be implanted into different pulmonary vein during the same procedure.

In some embodiments, the delivery catheter is constructed of sufficiently flexible material to allow insertion through the tortuosity imposed by the patient's vascular system.

In some embodiments, the delivery device shaft being constructed of sufficiently flexible material to allow insertion through lumen of the delivery catheter, either during insertion of the delivery catheter or inserted through lumen after the delivery catheter is positioned within the pulmonary vein.

An additional aspect of the disclosure provides an implant device for disrupting electrical signals traveling along a pulmonary vein by bidirectional stretching, the implant device comprising a radial expandable ring or coil configured to deploy within the pulmonary vein and deliver a force against pulmonary vein wall to provide bidirectional stretching of the pulmonary vein wall in the radial direction and axial direction, wherein bidirectional stretching creates a two-step biological response in the pulmonary vein wall to promote cellular decoupling, comprising an acute response is caused by pressure-induced apoptosis inhibiting chemical exchange of sodium/calcium and disrupting focal electrical wave propagation, and second, a biological response for chronic or long-term isolation/denervation is provided by causing focal endothelial cell proliferation at the implant site.

An additional aspect of the disclosure provides an implant device for disrupting electrical signals traveling along a pulmonary vein by bidirectional stretching, the implant device comprising a radial expandable ring or coil configured to deploy within the pulmonary vein and deliver a force against pulmonary vein wall to provide bidirectional stretching of the pulmonary vein wall in the radial direction and axial direction, wherein bidirectional stretching causes a first effect of stretching and micro-tears resulting in the creation of scar tissue formation, the scar tissue at least partially blocking and/or disrupting electrical conduction along the first pulmonary vein, and a second effect of outward pressure causing the myocytes to be compressed, reducing their ability to perform normal function.

An additional aspect of the disclosure provides a method of treating an arrhythmia by bidirectional stretching, comprising, selecting a patient, choosing a first implant device for insertion into a first pulmonary vein of the patient, inserting an implant device delivery catheter into the patient, wherein the implant delivery catheter comprises a distal end and a distal portion and the first implant device is positioned in the distal portion, advancing the distal portion of the implant delivery device into the first pulmonary vein, positioning the first implant device relative to the first pulmonary vein near the ostium and or left atrial—pulmonary vein junction, deploying the first implant device within the first pulmonary vein near the ostium and or left atrial—pulmonary vein junction, radially expanding to a diameter larger than the pulmonary vein, the first implant device to cause a first effect of stretching and micro-tears resulting in the creation of scar tissue formation, the scar tissue at least partially blocking and/or disrupting electrical conduction along the first pulmonary vein, and a second effect of outward pressure causing the myocytes to be compressed, reducing their ability to perform normal function, and withdrawing the distal end of the implant delivery device from the first pulmonary vein.

In some embodiments, the first effect is selected from the group consisting of:
stretching of a portion of the pulmonary vein causing micro-tears in a portion of the pulmonary vein;
stretching of a portion of the pulmonary vein ostium causing micro-tears in a portion of the pulmonary vein ostium;
stretching of a portion of the left atrial—pulmonary vein junction causing micro-tears in a portion of the left atrial—pulmonary vein junction; and
combinations thereof.

In some embodiments, the second effect is selected from the group consisting of:
outwards pressure applied to the pulmonary vein;
outwards pressure applied to the pulmonary vein ostium; and
outwards pressure applied to the left atrial—pulmonary vein junction; and combinations thereof.

### BRIEF DESCRIPTION OF THE DRAWINGS

FIG. 1 shows a percutaneous (interventional) deployment system for treating a heart arrhythmia, such as atrial fibrillation

FIG. 2A is a flow chart showing one method for using the system of FIG. 1.

FIG. 2B schematically illustrates a delivery device situating an implant within the pulmonary vein of a heart.

FIG. 2C schematically illustrates the implant device situated at the OS of a pulmonary vein.

FIGS. 3A, 3B, 3C show one embodiment of an expandable implant in an expanded state after being radially expanded using a deployment device.

FIGS. 4A-D shows one embodiment of a delivery device having multiple implants for use with a single delivery catheter.

FIGS. 5A-5C shows a delivery device configured to deliver one or more implants into one or more pulmonary veins using a delivery control rod.

FIG. 6 shows a dilator catheter having expansion portion with multiple dilator arms to apply force to a pulmonary vein wall and/or expand an implant.

FIG. 7 shows an ultrasound measurement device having an array of ultrasound transducers for measuring measure the diameter and/or overall dimensions of a pulmonary vein or ostium.

FIGS. 8A and 8B show two embodiments of dilation catheters. FIG. 8A showing a cage/basket type expansion portion with a plurality of expandable arm. FIG. 8B showing an expansion portion as an inflatable balloon.

FIG. 9 shows one embodiment of a pulmonary vein measurement device having a loop or coil device for measuring the size of a pulmonary vein.

FIGS. 10A-10D show a ratcheting implant delivery device.

FIG. 11 shows a tool for expanding or contracting a “hose clamp” implant within a pulmonary vein.

FIGS. 12A-12F show one embodiment of a braided cage implant.

FIGS. 13A-13B show an adjustable braid implant having a therapy portion and an anchor portion.

FIGS. 14A-14F show simulation results of a therapeutic stretching of a pulmonary vein with an implant.

FIG. 15 shows one embodiment of a braided implant having a pulmonary sleeve or covering with an ostium portion and a pulmonary vein portion.

FIG. 16 shows one embodiment of a braided implant having a pulmonary sleeve or covering with a pulmonary vein portion.

FIG. 17 shows embodiments of delivery catheter designs.

FIG. 18 shows one embodiment of a balloon expanding delivery catheter and implant.

### DETAILED DESCRIPTION

Embodiments of the invention will now be described with reference to the figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein. The words proximal and distal are applied herein to denote specific ends of components of the instrument described herein. A proximal end refers to the end of an instrument nearer to an operator of the instrument when the instrument is being used. A distal end refers to the end of a component further from the operator and extending towards the surgical area of a patient and/or the implant.

The present invention provides implants, systems and methods for creating a region/area of non-conductive tissue at the pulmonary vein ostium and/or within the pulmonary vein to disrupt (e.g., stop, slow, otherwise impact, etc.) electrical signals and/or nerves traveling along the pulmonary vein. The implant provides mechanical energy against tissue, eliminating the electrical refractory process of the myocytes on a cellular level and inhibiting the chemical reaction at the focal site of the implant, thus rendering the tissue electrically inert at the contact point of the implant and creating focal necrosis in a line of block. This can be accomplished by bidirectional stretching of the vessel or lumen in the radial direction and axial (or longitudinal) direction by the implant.

The bidirectional stretching of the vessel or lumen creates a two-step biological response to promote cellular decoupling. First, an acute response is caused by pressure-induced apoptosis inhibiting chemical exchange of sodium/calcium and disrupting focal electrical wave propagation. Second, a biological response for chronic or long-term isolation/denervation is provided by causing focal endothelial cell proliferation at the implant site. As a result, the implant causes disruption of electrical or neural conduction along the vessel or body lumen. In some embodiments, such an initial acute response can be followed by a chronic response, in which fibrogen (e.g., collagen fibers) can fill the interstitial spaces that have been increased due to strain-based myocyte displacement. In turn, in some embodiments, this can create a long-term (e.g., permanent) non-conducting modification of the substrate of the vessel or other body lumen. As noted above, such a stretching (e.g., in the radial and axial directions) can be accomplished using any of the implants disclosed herein.

In some embodiments, the implant is configured to apply and maintain a radial force or substantially radial force at the pulmonary vein ostium and/or along the circumference of the pulmonary vein in which it is implanted. The radial force imparted from the implant on the pulmonary vein wall is found to electrically isolate the pulmonary vein.

While the implants, systems and methods are described in relation to treatment of the pulmonary vein, they may also be used in other parts of the body, such as a renal artery, another type of blood vessel, an airway, a urinary tract vessel, a lumen of the gastroenterological system, etc., as desired or required for the treatment of a particular disease or condition.

### Deployment System

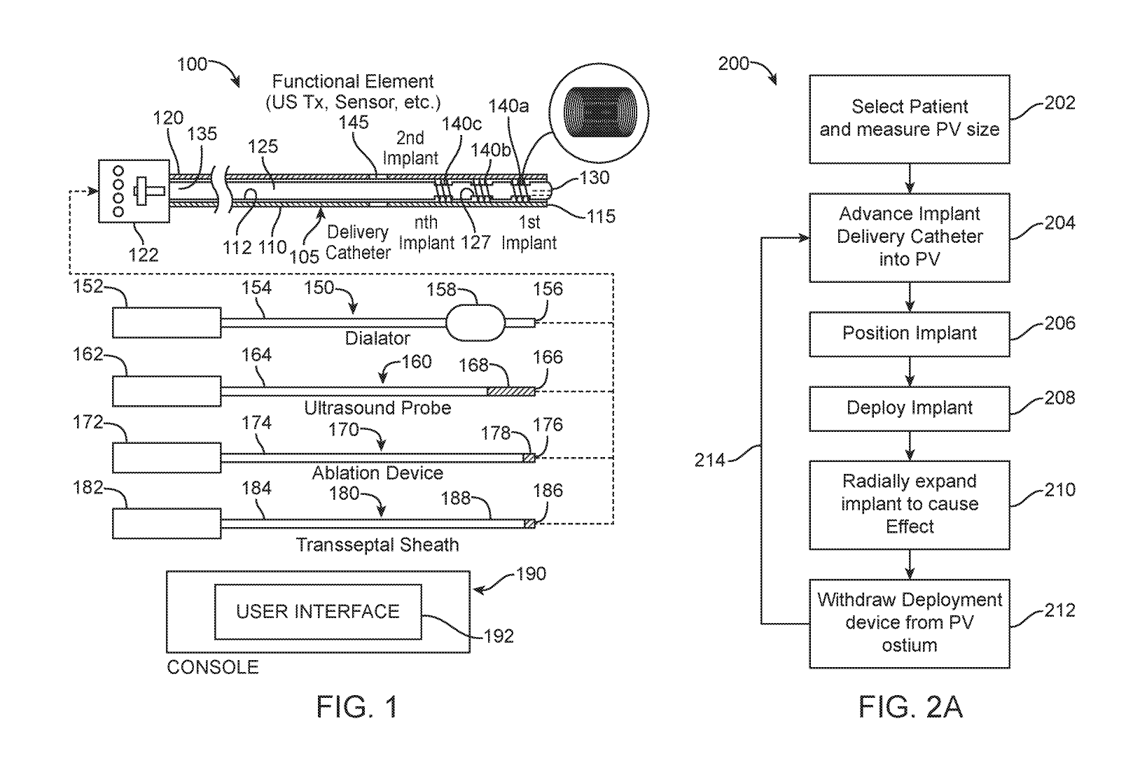

FIG. 1 shows one embodiment of a percutaneous (interventional) deployment system 100 configured to provide and retain a therapeutic stretch for electrically isolating a pulmonary vein of a patient. The therapeutic stretch of the pulmonary vein and ostium wall may significantly diminish or eliminate ion exchange between cells in the pulmonary vein and ostium wall. The therapeutic stretch may include bidirectional stretching of the ostium wall and/or pulmonary vein to create micro-tears to disrupt (e.g., stop, slow, otherwise impact, etc.) electrical signals and/or nerves traveling along the pulmonary vein.

System 100 includes a delivery catheter 105, a delivery device 125 and one or more implants 140a, 140b, 140c. Delivery catheter 105 is constructed and arranged for insertion into a body location, such as the pulmonary vein. Delivery catheter 105 includes a shaft 110 having a distal end 115 and proximal end 120 constructed of sufficiently flexible material to allow insertion through the tortuosity imposed by the patient's vascular system. Shaft 110 includes a lumen 112 traveling from the proximal end 115 to the distal end 120. Lumen 112 is constructed and arranged to allow delivery device 125 to be slidingly received by lumen 112. Lumen 112 can be further configured to slidingly receive additional catheters or other elongate devices, such as a dilator 150, an imaging device 160, an ablation device 170 and a transseptal sheath 180.

Implant 140 may have a ring or coil design, and may be made from a variety of materials, such as: stainless steel, Elgiloy, nickel, titanium, nitinol, polymers, shape memory polymers, bioresorbable materials, etc. Implant 140 is configured to provide a continuous pressure against the pulmonary vein and ostium wall. Implant 140 may be resiliently biased (i.e. self-expanding), plastically deformable (i.e. balloon expandable) and/or include both resiliently biased and plastically deformable sections. Implant 140 may be used with a dilator for implantation, such as balloon dilator 150. Implant 140 may be a two part implant having differing materials or construction, such as combination of metal and non-metal parts. This may allow implant 140 to have a conductive portion and a non-conductive portion. Implant 140 may be bioresorbable and made from a bioresorbable material, such as poly-L-lactic acid, PLLA, PLDA, PCL-PLA blends, or any bioresorbable polymer with suitable material properties for radial strength retention and degradation. Implant 140 may have a coating. In some embodiments, the coating may create scar tissue. In other embodiments, the coating may be a drug coating.

Shaft 110 may include one or more functional elements 145. In one embodiment, functional elements 145 are configured to provide imaging with one or more ultrasound transducers, and provide imaging information to a console 190. In another embodiment, functional elements 145 are configured to provide heat using one or more electrodes, for example, using radiofrequency (RF), to a deployed implant. The generated heat facilitates electrical isolation of the PV from the chamber.

System 100 may also include a handle 122 positioned on shaft's proximal end 115. Handle 122 may include a controller for advancement and deployment of implant one or more implants 140a, 140b, 140c. Handle 122 may control dilating a balloon, if included. Handle 122 may provide force feedback of applied force during a procedure.

Delivery device 125 includes a shaft 127 having a distal end 130 and proximal end 135 constructed of sufficiently flexible material to allow insertion through lumen 112 of the delivery catheter 105. One or more implants may be positioned proximate the distal end 130. There are three implants 140a, 140b, 140c shown in the figure. Delivery device 125 may be positioned within lumen 112 during insertion of the delivery catheter 105, or may by inserted through lumen 112 after delivery catheter 105 is positioned within the pulmonary vein. In some embodiments, the implants are self expanding, so as distal end 130 exits distally from lumen 112, exposing implant 140, implant 140 may expand away from shaft 127. In some embodiments, delivery device 125 may include one or more balloons positioned under the implants 140 for balloon expandable deployment of the implants. Delivery device 125 is configured to deliver one implant at a time, so that multiple implants may be implanted into a single pulmonary vein, or individual implants may be implanted into different pulmonary veins during the same procedure. For example, the three implants 140 shown in the figures may be implanted into one, two or three pulmonary veins.

In some embodiments, system 100 may further include a dilator device 150 configured to be slidingly received by lumen 112 of delivery catheter 105. Dilator device 150 includes a handle 152 fixedly attached to a flexible shaft 154 having a distal end 156 and an expansion portion 158. Expansion portion 158 may be an inflatable balloon. Handle 152 may control dilating the balloon. Expansion portion 158 is configured to apply force to the pulmonary vein wall, either before or after placement of implant 140. The applied force enhances stretching and/or micro tearing of the tissue. The stretching may be between 1 mm and 10 mm. The stretching may increase the inside diameter of the pulmonary vein 1.5 to 2.5 times the original size. Expansion portion 158 may also expand the pulmonary vein in multiple steps. Dilator device 150 may be integrated with delivery device 125.

In some embodiments, system 100 may include an imaging device 160 configured to be slidingly received by lumen 112 of delivery catheter 110. Imagining device 160 includes a handle 162 fixedly attached to a flexible shaft 164 having a distal end 166 and an imaging portion 168. Imaging portion 168 may be fluoroscope, x-ray or ultrasound. Imaging portion 168 may include one or more electrodes or one or more ultrasound transducers mounted to shaft 164 configured to provide information to a console 190. Console 190 may include an output device 192, such as a visual display, to provide a visual image of the patient's anatomy.

In some embodiments, system 100 may further include an ablation device 170 configured to be slidingly received by lumen 112 of delivery catheter 110. Ablation device 170 includes a handle 172 fixedly attached to a flexible shaft 174 having a distal end 176 and at least one ablation element 178. Ablation element 178 is constructed and arranged to deliver energy to tissue when attached to a source of energy, such as console 190.

In some embodiments, system 100 may include a transseptal sheath 180 having a handle 182 fixedly attached to a flexible shaft 184 having a distal end 186 and lumen 188. Lumen 188 is constructed and arranged to allow delivery catheter 105 to be slidingly received by lumen 188. Lumen 188 can be further configured to slidingly receive additional catheters or other elongate devices, such as dilator 150, imaging device 160 and ablation device 170.

### Method and Placement

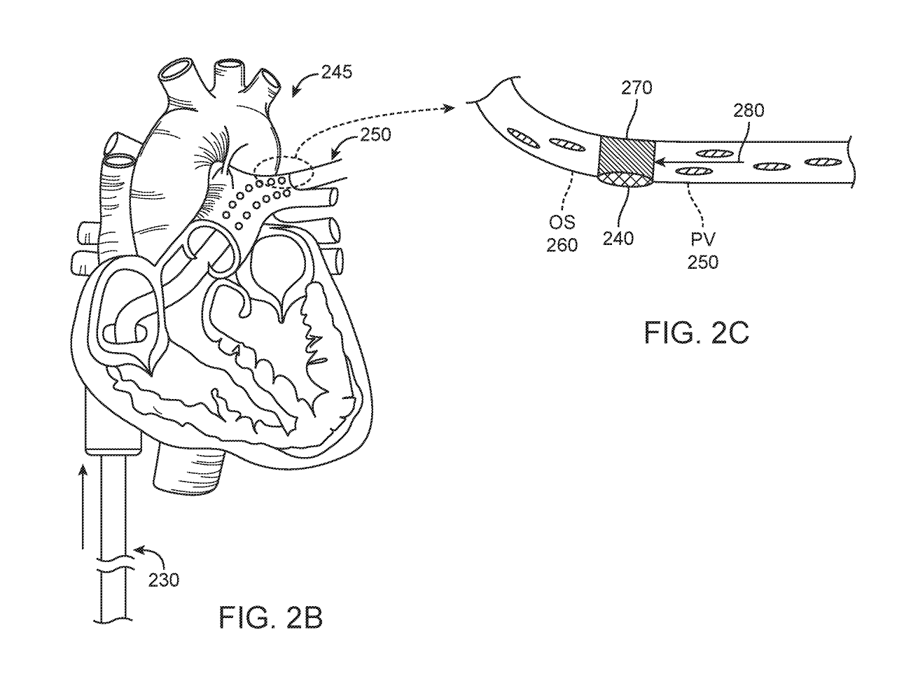

FIG. 2A is a flow chart showing one method 200 for using system 100 of the present invention. The steps include:
STEP 202: Selecting a patient having one or more pulmonary veins to be treated. Measure the one or more pulmonary veins to select the appropriate implant size and achieve desired amount of oversize (stretch ratio) of the target pulmonary vein.
STEP 204: Advancing an implant delivery catheter into a pulmonary vein ostium.
STEP 206: Positioning an implant within the pulmonary vein.
STEP 208: Deploying the implant with an implant deployment device.
STEP 210: Radially expanding the implant to cause a therapeutic effect on the pulmonary vein using the deployment device or a separate dilating catheter (e.g., balloon dilator 150).
STEP 212: Withdrawing deployment device from pulmonary vein ostium.
STEP 214: Another implant may be implanted in the same or different pulmonary vein ostium by returning to STEP 204.

In some embodiments, the steps may also include identify the myocardial sleeve using a mapping catheter, such as a circular mapping catheter or a basket catheter, and placing the implant such that it is contacting the myocardial sleeve.

FIG. 2B illustrates one embodiment of a delivery system 230 delivering an implant 240 to a desired anatomical location (e.g., in the left atrium of the heart 245, and in particular into a pulmonary vein (PV) 250, another vessel or portion of a subject, etc.). Implant 240 can be any desired shape and be deployed in many different manners from the distal end of the delivery system. FIG. 2C illustrates one embodiment of an implant 240 within pulmonary vein 250. Implant 240 is placed at the ostium (OS) 260 or adjacent the OS in the pulmonary vein 250 and then be deployed. Once delivered, the implant 240 can exert pressure against an inner wall of the pulmonary vein 250, in this case, near OS 260. As discussed herein, such pressure creates a conductive block 270 and isolates the pulmonary vein from the atrium. By placing an implant in the pulmonary veins, aberrant electrical signals 280 emanating from the pulmonary vein may be effectively blocked from reaching the heart 245. Such pulmonary vein isolation is believed to be highly accurate and therapeutic in treating atrial fibrillation. In some embodiments, the shape of the outer surface of the implant is shaped, sized and otherwise configured so as to not penetrate the adjacent tissue, while still exerting the necessary pressure to induce the necessary physiological response.

### Ratcheting Implant

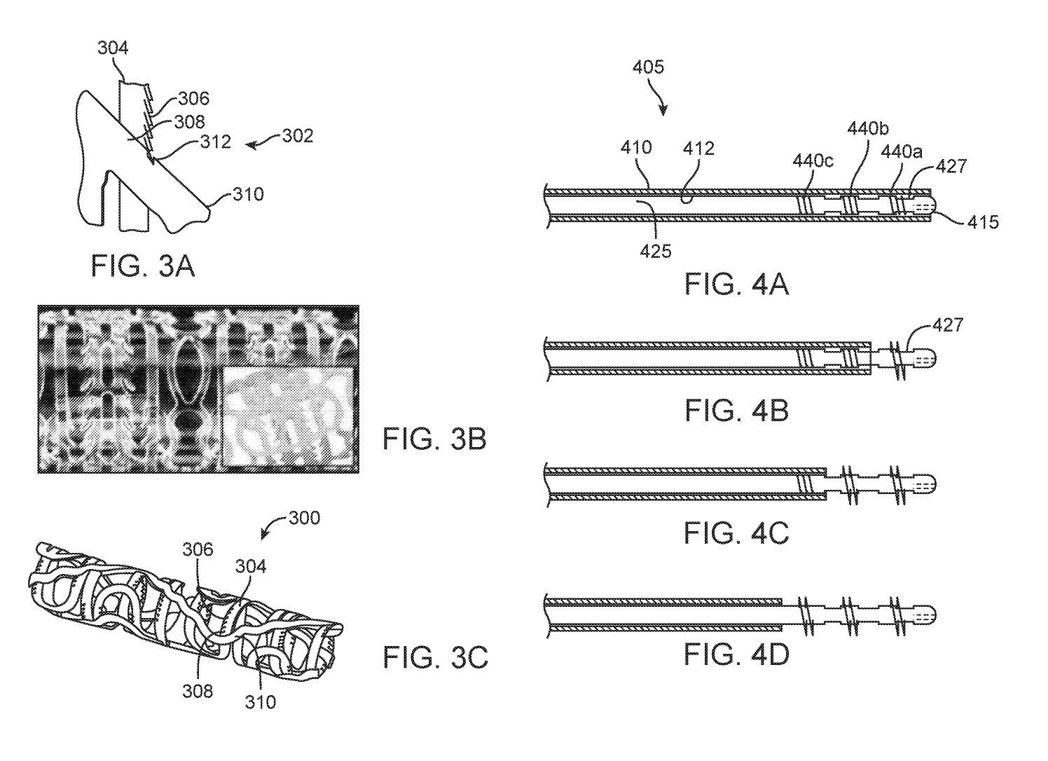

FIGS. 3A, 3B, 3C show one embodiment of an expandable implant 300 in an expanded state after being radially expanded using a deployment device, such as delivery device 125, or dilating catheter, such as balloon dilator 150. Implant 300 includes a ratcheting expansion feature 302 including a plurality of straps 304 having a plurality of teeth 306, and a plurality of receiver slots 308 within backbone members 310 configured to receive straps 304. Straps 304 and slots 308 are directed in a perpendicular direction relative to the implant axis. During expansion, straps 304 pass slidably through receiver slots 308 in adjacent backbones 308, moving circumferentially farther away from each slot 308. Each tooth 306 can be deflected on entry into receiver slot 308, and upon leaving the receiver slot 308, tooth 308 returns to its normal position and engages a stop 312 of receiver slot 308. Such movement results in unidirectional slidable movement of strap 304 through receiver slot 308, locking implant 300 in the expanded state.

### Delivery Device with Multiple Implants

FIGS. 4A-D shows one embodiment of a delivery device 425 having multiple implants 440a, 440b, 440c for use with a single delivery catheter 405. In the figures, three implants are shown, but any number of implants may be delivered. The implants may be delivered and implanted in one pulmonary vein (multiple implants in a single pulmonary vein) or multiple pulmonary veins. During delivery, shaft 410 acts as a sheath and implants 440 exit from distal end 415. For a self-expanding implant, retraction of the sheath causes resiliently biased expansion of the implant. For a non-self-expanding implant, delivery device 425 may include a deployment device or a separate dilating catheter (e.g., balloon dilator 150) may be used.

FIG. 4A shows three implants 440a, 440b, 440c positioned on delivery device 425 within lumen 412 of delivery catheter 405. In the embodiment shown, delivery device 425 includes reduced diameter areas 427 for the implants to nest in. FIG. 4B shows retraction of sheath 410 and delivering of first implant 404a. FIG. 4C shows further retraction of sheath 110 and delivery of second implant 404b. FIG. 4D shows still further retraction of sheath 410 and delivery of third implant 404c. In between delivery of each implant, delivery catheter 405 may be repositioned within the same pulmonary vein or moved to a different pulmonary vein. Also between deliveries, sheath 410 may be extended back to its original position for moving delivery catheter 405 to the new location.

### Delivery Device with Multiple Implants and Delivery Control Rod

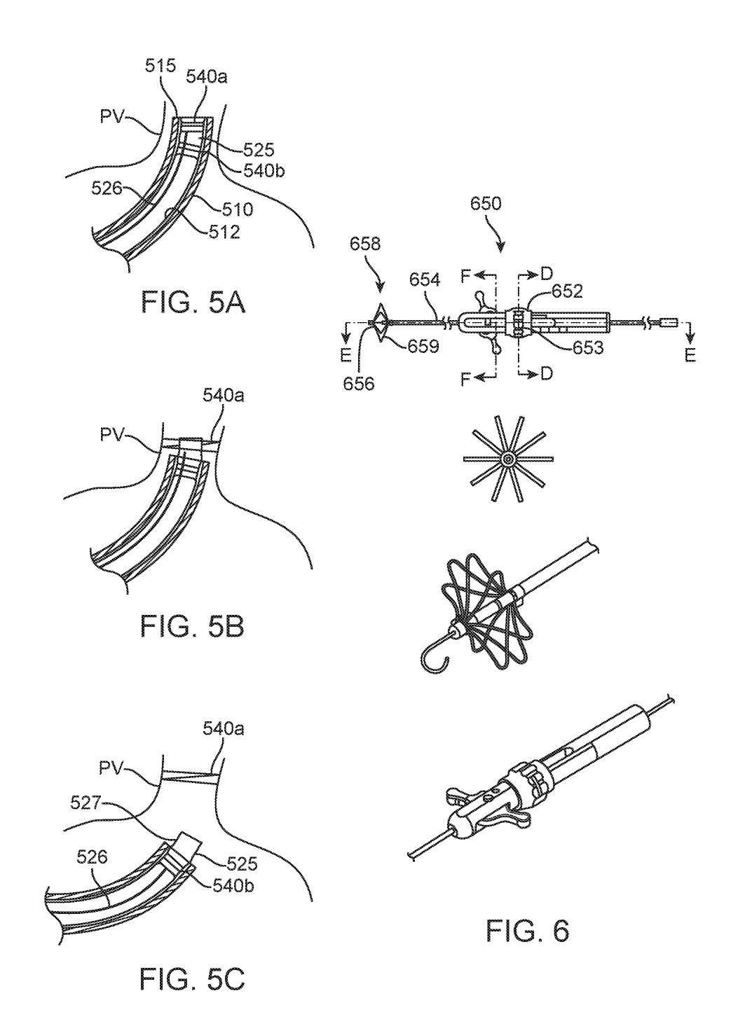

FIGS. 5A-5C shows a delivery device 525 configured to deliver one or more implants through lumen 512 of sheath 510 into one or more pulmonary veins PV using a delivery control rod 526. While two implants 540a, 540b are shown in the figures, any number of implants may be delivered. In the embodiment shown, delivery device 525 includes reduced diameter areas 527 for the implants to nest in. Control rod 526 is configured to “pushes out” implants 540a, 540b from distal end 515 of sheath 510. Control rod 526 may also be configured to manipulate implants 540a, 540b into position. Control rod 526 may also be configured to recapture implants 540a, 540b to reposition or remove them. For example, control rod 526 may include a “grasper” configured to engage the implants for recapture.

One method for using delivery device 525 include the steps of:
STEP 502: Selecting a patient having one or more pulmonary veins PV to be treated. Measure the one or more pulmonary veins to select the appropriate implant size and achieve desired amount of oversize (stretch ratio) of the target pulmonary vein.
STEP 504: Advancing an implant delivery catheter into a pulmonary vein ostium.
STEP 506: Positioning an implant within the pulmonary vein, proximate the ostium.
STEP 508: Advancing an implant through the lumen of the delivery device into the pulmonary vein using a delivery control rod.
STEP 510: Deploying the implant with an implant deployment device.
STEP 512: Radially expanding the implant to cause a therapeutic effect on the pulmonary vein using the deployment device or a separate dilating catheter.
STEP 514: Repositioning implant delivery catheter within the same pulmonary vein to deliver another implant, or withdrawing implant delivery catheter from pulmonary vein.
STEP 516: Another implant may be also be implanted in a different pulmonary vein ostium by returning to STEP 504.

### Dilator Catheter Includes Multiple Arms (e.g. Non-Balloon Option)

FIG. 6 shows a dilator catheter 650 configured to be slidingly received by lumen 112 of delivery catheter 105. Dilator catheter 650 includes a handle 652 coupled to a flexible shaft 654 having a distal end 656 and an expansion portion 658 having multiple dilator arms 659. A knob 653 on handle 652 translates an internal shaft which radially expands dilator arms 659. Knob 653 can be rotated to lock dilation. Dilation amount can be measured by linear translation distance of knob 653 (control).

Expansion portion 658 is configured to apply force to expand an implant within the pulmonary vein with enough pressure against the pulmonary vein wall to achieve electrical isolation. If needed, expansion portion 658 may be rotated between expansions (e.g. 90°). This may be repeated as needed until electrical isolation is achieved. Expansion portion 658 may also be used to apply a force to the pulmonary vein wall, either before or after expansion of the implant. The applied force enhances bidirectional stretching and/or micro tearing of the tissue. The stretching may be between 1 mm and 10 mm. The stretching may increase the inside diameter of the pulmonary vein 1.5 to 2.5 times the original size. Expansion portion 658 may also expand the pulmonary vein in multiple steps, and may be rotated between expansions (e.g. 90°).

### Ultrasound Measurement Device

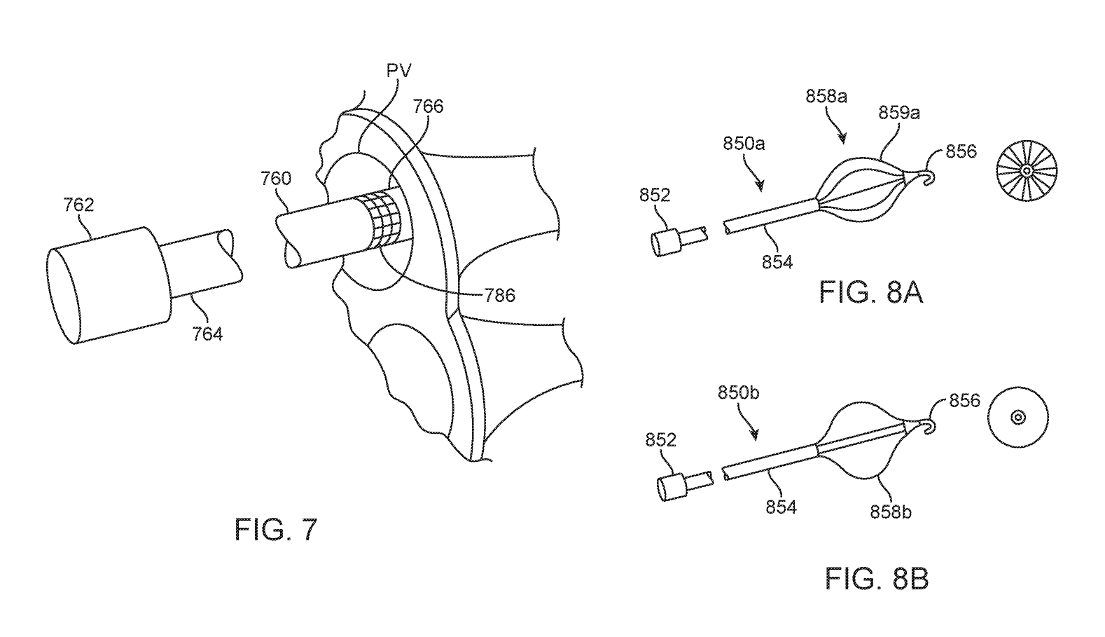

FIG. 7 shows an ultrasound measurement device 760 entering a pulmonary vein (PV) ostium. Ultrasound measurement device 760 is configured to be slidingly received by lumen 112 of delivery catheter 105. Dilator catheter 760 includes a handle 762 coupled to a flexible shaft 764 having a distal end 766 and a circumferential array of ultrasound transducers 768. In some embodiments, the array may be a piezoelectric micromachined ultrasonic transducers (pMUTs) array. Ultrasound measurement device 760 may be used to measure the diameter and/or overall dimensions of the PV ostium either pre-deployment or post-deployment of the implant. Ultrasound transducers 768 may be configured to provide information to console 190. Optionally, the array of ultrasound transducers may be functional element 145 of delivery catheter 105.

### Dilator Catheters

FIGS. 8A and 8B show two embodiments of dilation catheters 850a, 850b configured to be slidingly received by lumen 112 of delivery catheter 105. Each dilation catheter 850a, 850b includes a handle 852 fixedly attached to a flexible shaft 854 having an atraumatic distal tip 856 and an expansion portion 858a, 858b. In some embodiments, expansion portion 858 may be configured to apply force to the pulmonary vein wall, either before or after placement of an implant. FIG. 8A shows a cage/basket type expansion portion 858a with a plurality of expandable arms 859a, and handle 852 may control expansion of the expandable arms 859a during dilation. FIG. 8B shows expansion portion 858b as an inflatable balloon, and handle 852 may control dilatation of balloon 858b. In some embodiments, inflatable balloon 858b may be a shaped balloon configured to apply pressure in specific pattern (narrow ring). In some embodiments, expansion portion 858a, 858b is configured to apply force to expand an implant within the pulmonary vein with enough pressure against the pulmonary vein wall to achieve electrical isolation. In some embodiments, expansion portion 858a, 858b is configured to apply force to the pulmonary vein wall, either before or after placement of an implant. In some embodiments, expansion portion 858a, 858b is configured to apply force to the pulmonary vein wall before placement of an implant and apply force to expand the implant within the pulmonary vein, and optionally, apply force to the pulmonary vein wall after placement of the implant.

### Pulmonary Vein Measurement Device

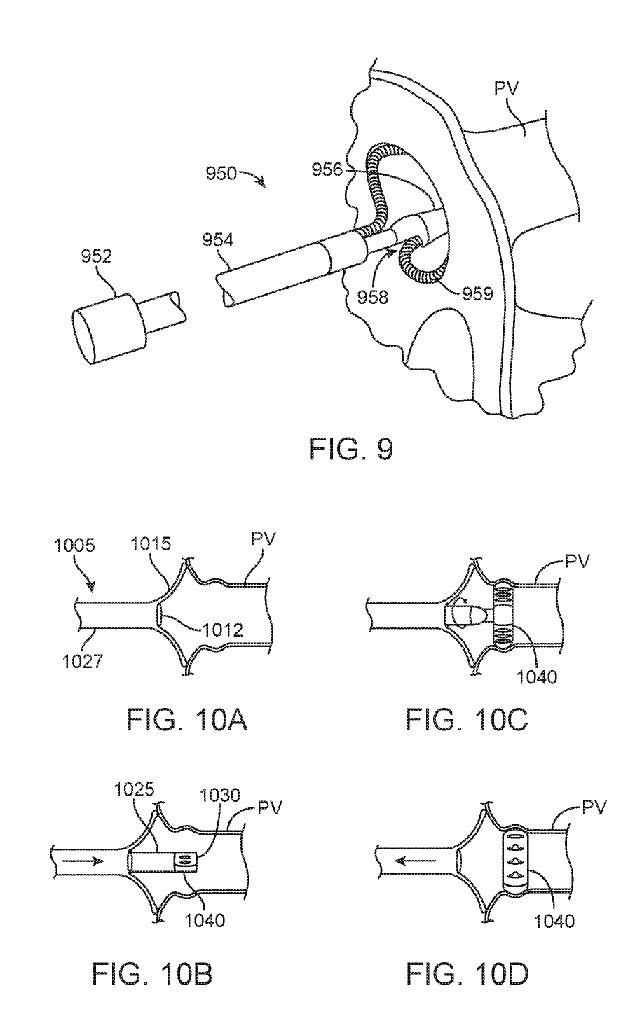

FIG. 9 shows one embodiment of a pulmonary vein measurement device 950 with a handle 952 fixedly attached to a flexible shaft 954 having a distal end 956 and a measurement system 958 comprising a loop or coil 959 for measuring the size of a pulmonary vein PV, such as pulmonary vein diameter, axis, etc. Controls on handle 952 are configured to measure the amount of “uncoiling” of the loop 959 within the pulmonary vein to determine the pulmonary vein size. The measurement may be used to determine appropriate size of implant to be used and/or dilation amount for the implant.

### Ratcheting Implant Delivery Device

FIGS. 10A-10D show a ratcheting implant delivery device 1025 having a shaft 1027 and distal end 1030, and a ratcheting implant 1040 being implanted in a pulmonary vein PV. Shaft 1027 is constructed of sufficiently flexible material to allow insertion through a lumen 1012 of a delivery sheath 1005. Delivery sheath 1005 may include placement guides 1015 configured to hold the delivery sheath 1005 in position against the pulmonary vein ostium. FIGS. 10A-10D show four steps of use. FIG. 10A shows delivery sheath 1005 and placement guides 1015 positioned in place against the pulmonary vein ostium. FIG. 10B shows delivery device 1025 exiting a distal end of delivery sheath 1005 and positioning ratcheting implant 1040 within the pulmonary vein PV. FIG. 10C shows expansion of ratcheting implant 1040 using a control mechanism configured to rotate and expand ratcheting implant 1040 against the pulmonary vein wall. FIG. 10D shows expanded ratcheting implant 1040 left in place after delivery device 1025 removed from delivery sheath 1005.

### Tool For Manipulating Implant

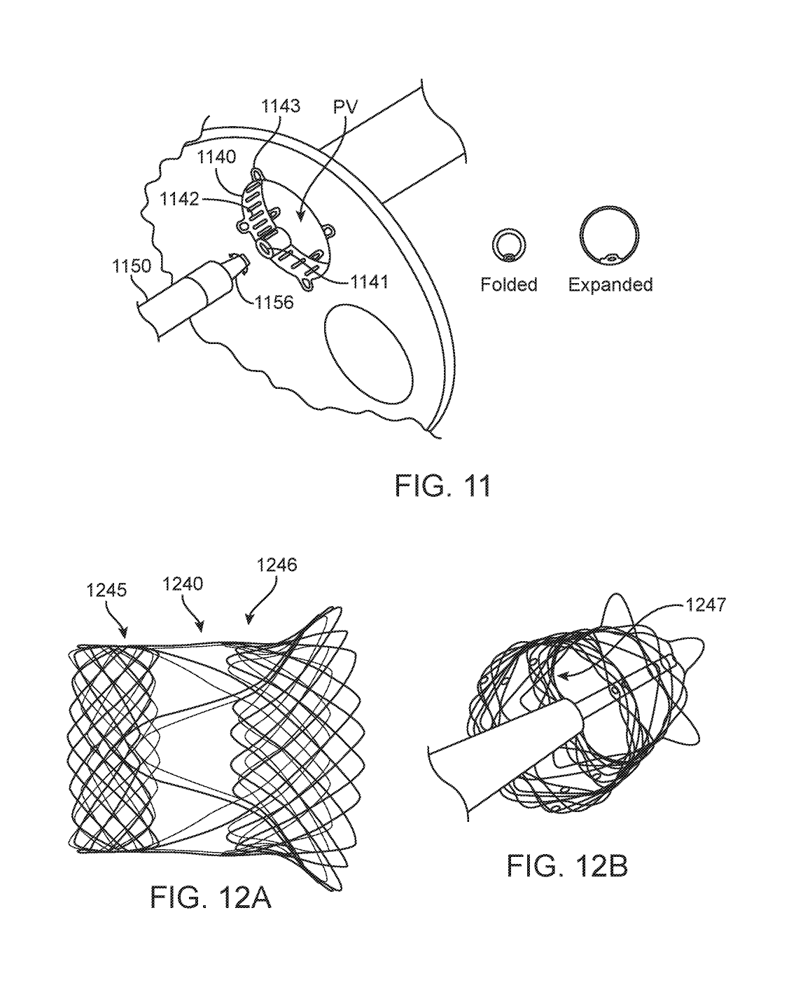

FIG. 11 shows a tool 1150 for expanding or contracting an implant 1140 within a pulmonary vein PV. Implant 1140 is similar to a “hose clamp”, having a gear 1141 configured to engage slots 1142. A distal end 1156 of tool 1150 is configured to engage gear 1141. Rotation 155 of distal end 1156 causes expansion or contraction of implant 1140 within the pulmonary vein. Implant 1140 may also include one or more stabilizers 1143. Tool 1150 and implant 1140 may be combined in an all-in-one implant/dilator configuration and may be implanted as shown in FIGS. 10A-10D.

### Braided Cage Design of Implant

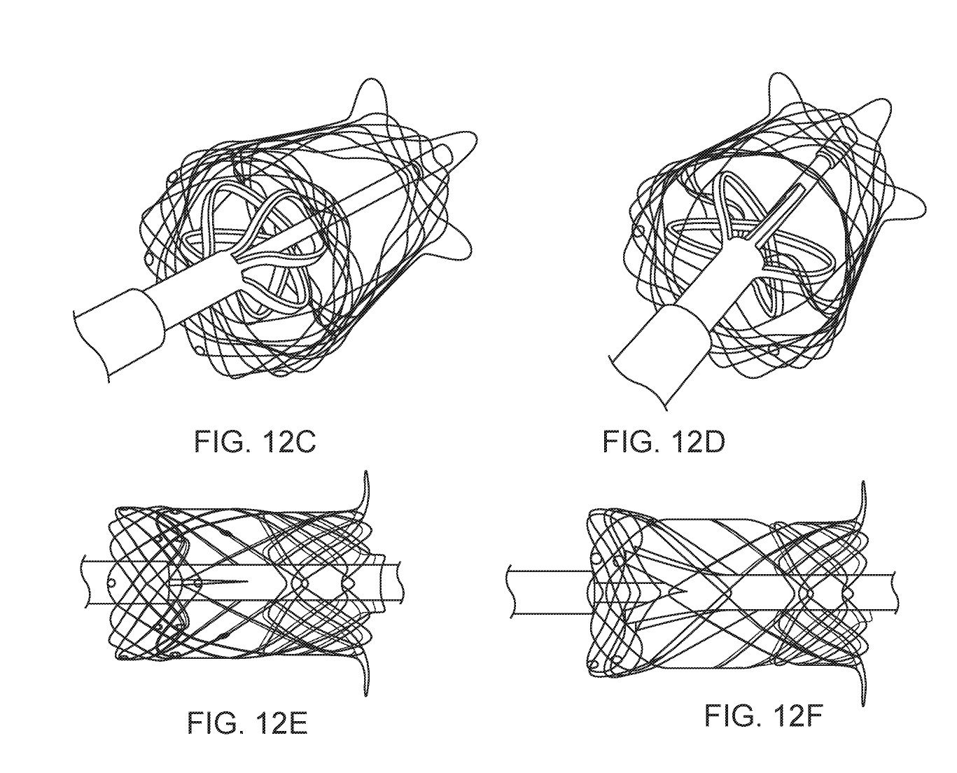

FIGS. 12A-12F show embodiments of a braided cage implant 1240 having a therapy portion 1245 and an anchor portion 1246. Therapy portion 1245 is configured to apply a force to the pulmonary vein wall with braided cage implant 1240 to achieve bidirectional stretching and/or micro tearing of the pulmonary vein wall tissue to provide electrical isolation. FIG. 12A shows braided cage implant 1240 as a braided NiTi wire with shape-set structure and multiple interconnected scaffold zones that provide locally optimized radial force, radial strength, anchoring, durability, etc. FIG. 12B shows a novel tensioned-suture active truss 1247 that delivers therapeutic over-stretch during deployment. FIGS. 12C-F show expansion of the braided cage implant 1240 during deployment to achieve therapeutic over-stretch, including bidirectional stretching and/or micro tearing, of the pulmonary vein wall tissue to provide electrical isolation.

### Implant with Adjustable Braid

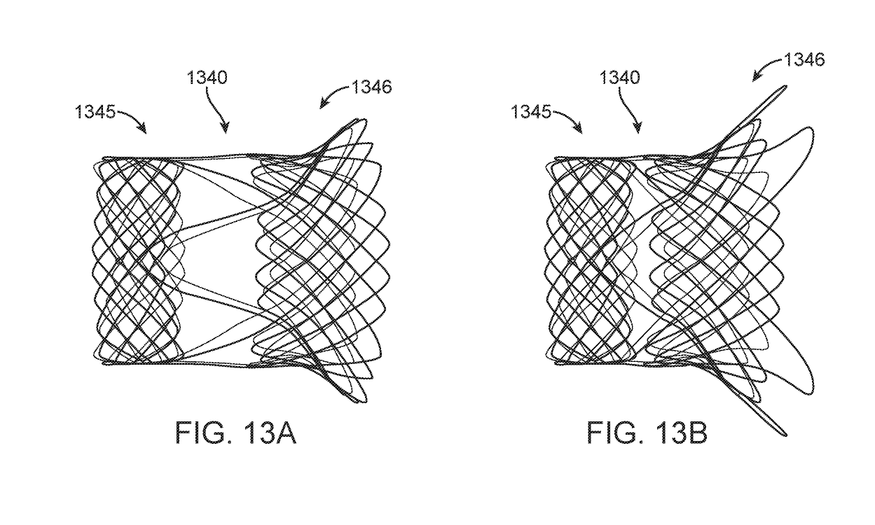

FIGS. 13A-13B show an adjustable braid implant 1340 having a therapy portion 1345 and an anchor portion 1346. Adjustable braid implant 1340 is made of a braided wire that a user can adjust to shape either pre or intra procedure. Adjustable braid can also dilate therapy ring.

### Simulation Results

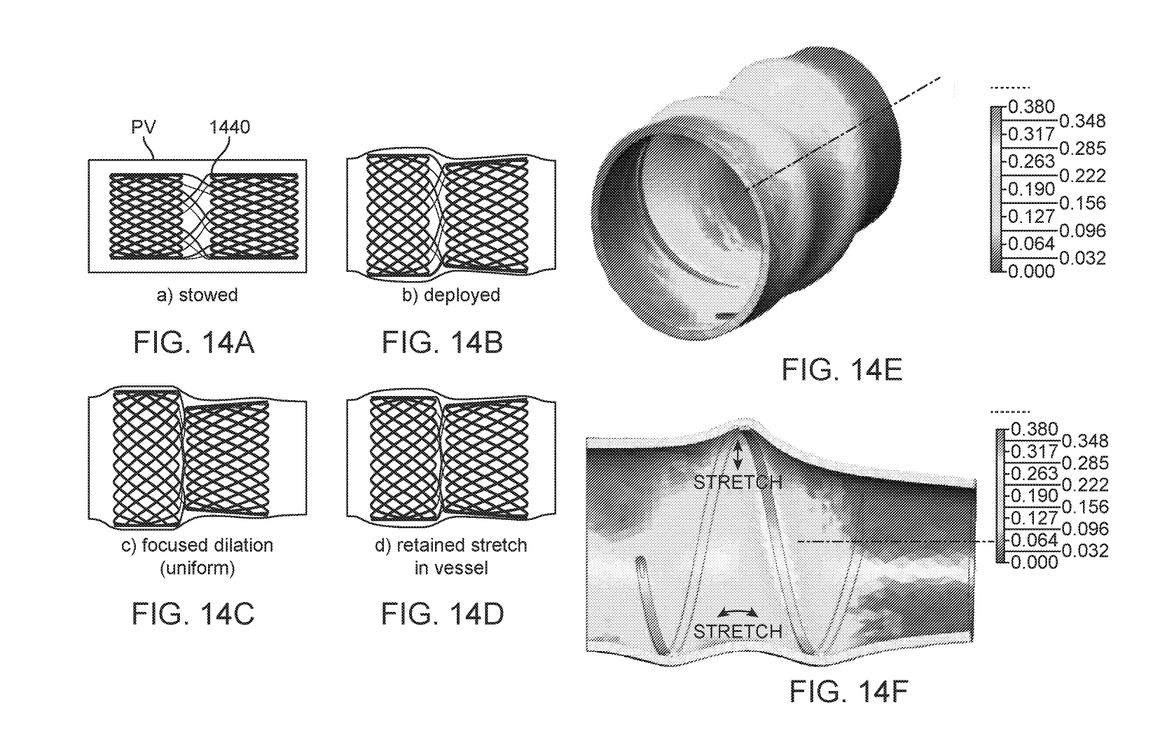

FIGS. 14A-14F show simulation results of a therapeutic stretching of a pulmonary vein PV with an implant 1440 to achieve electrical isolation. FIG. 14A shows implant 1440 in a stowed position within the pulmonary vein. FIG. 14B shows implant 1440 in a deployed position providing bidirectional stretching and/or micro tearing of the tissue and dilating the pulmonary vein. FIG. 14C shows focused dilation of the pulmonary vein by implant 1440. FIG. 14C shows the retained stretch of implant 1440 within the pulmonary vein to maintain electrical isolation. FIGS. 14E-14F show additional FEA results illustrating radial stretching like but also illustrating that the implant can impart the desired effect on the PV tissue without making direct contact, in addition to affecting the PV substrate via direct contact.

### Implants Having a Pulmonary Sleeve

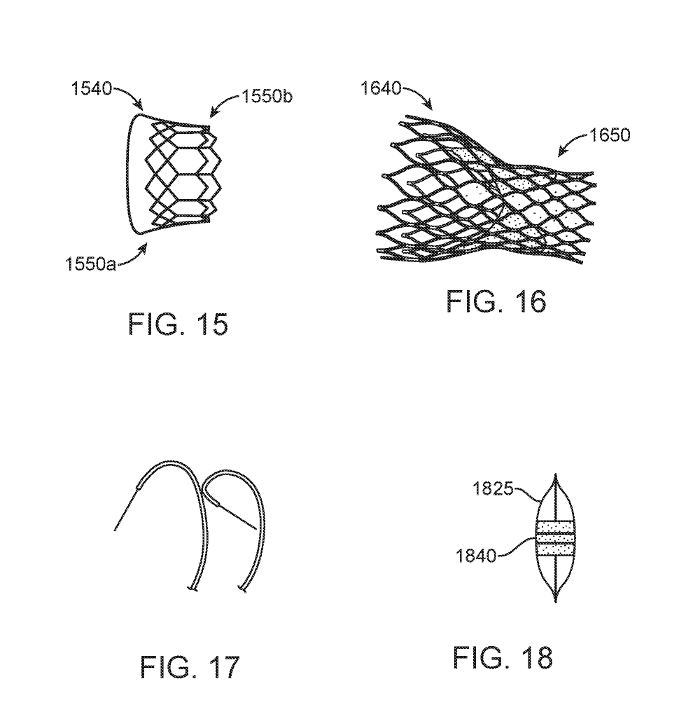

FIGS. 15 and 16 show embodiments of braided implants having a pulmonary sleeve or covering. The implants are configured to provide and retain optimized therapeutic stretch to a pulmonary vein and ostium. In FIG. 15, implant 1540 includes a pulmonary sleeve 1550 having an ostium portion 1550a to a pulmonary vein portion 1550b. In FIG. 16, implant 1640 includes a pulmonary sleeve 1650 having a pulmonary vein portion 1650.

### Delivery Catheter Designs

FIG. 17 shows embodiments of delivery catheter designs. Delivery catheter is constructed and arranged for insertion into a body location, such as the pulmonary vein. Delivery catheter includes a shaft having a distal end and proximal end constructed of sufficiently flexible material to allow insertion through the tortuosity imposed by the patient's vascular system. Shaft includes a lumen traveling from the proximal end to the distal end. Lumen is constructed and arranged to allow delivery devices to be slidingly received by lumen. Lumen can be further configured to slidingly receive additional catheters or other elongate devices, such as a dilator, an imaging device, an ablation device and a transseptal sheath.

### Expand Balloon within Implant

FIG. 18 shows one embodiment of a balloon expanding delivery catheter 1825 and implant 1840.

While the foregoing has described what are considered to he the best mode and/or other preferred embodiments, it is understood that various modifications can be made therein and that the invention or inventions can be implemented in various forms and embodiments, and that they can be applied in numerous applications, only some of which have been described herein. it is intended by the following claims to claim that which is literally described and all equivalents thereto, including all modifications and variations that fall within the scope of each claim.

1. A method of treating an arrhythmia, comprising:
selecting a patient;
choosing a first implant device for insertion into a first pulmonary vein of the patient;
inserting an implant device delivery catheter into the patient, wherein the implant delivery catheter comprises a distal end and a distal portion and the first implant device is positioned in the distal portion;
advancing the distal portion of the implant delivery device into the first pulmonary vein;
positioning the first implant device relative to the first pulmonary vein near the ostium and or left atrial—pulmonary vein junction;
deploying the first implant device within the first pulmonary vein near the ostium and or left atrial—pulmonary vein junction;
radially expanding to a diameter larger than the pulmonary vein, the first implant device to cause an effect selected from the group consisting of:
stretching of a portion of the pulmonary vein;
stretching of a portion of the pulmonary vein ostium;
stretching of a portion of the left atrial—pulmonary vein junction;
causing micro-tears in a portion of the pulmonary vein;
causing micro-tears in a portion of the pulmonary vein ostium;
causing micro-tears in a portion of the left atrial—pulmonary vein junction;
and combinations thereof; and
wherein the stretching and micro-tears resulting in the creation of scar tissue formation, the scar tissue at least partially blocking and/or disrupting electrical conduction along the first pulmonary vein;
withdrawing the distal end of the implant delivery device from the first pulmonary vein.

2. The method of claim 1, wherein radially expanding to a diameter larger than the pulmonary vein, the first implant device to cause an effect selected from the group consisting of:
outwards pressure applied to the pulmonary vein;
outwards pressure applied to the pulmonary vein ostium;
outwards pressure applied to the left atrial—pulmonary vein junction;
and combinations thereof; and
wherein the outward pressure causing the mycotes to be compressed, reducing their ability to perform normal function.

3. The method of claim 1, wherein radially expanding the first implant device includes holding open the first pulmonary vein at the larger diameter than normal without recoil.

4. The method of claim 1, wherein the first implant device includes multiple implants in the distal portion of the implant device delivery catheter being configured to deliver multiple implants into the first pulmonary vein, or individual implants into different pulmonary veins during the procedure

5. The method of claim 1, wherein implant device comprises a radial expandable ring or coil configured to deploy within the pulmonary vein and deliver a force against pulmonary vein wall to provide bidirectional stretching of the wall in the radial direction, causing pressure on the vein, and axial direction, causing stretching or tearing of the vein.

6. The method of claim 1, wherein radially expanding to the larger diameter larger than the pulmonary vein creates a two-step biological response in the pulmonary vein wall to promote cellular decoupling, comprising:
an acute response is caused by pressure-induced apoptosis inhibiting chemical exchange of sodium/calcium and disrupting focal electrical wave propagation; and
second, a biological response for chronic or long-term isolation/denervation is provided by causing focal endothelial cell proliferation at the implant site.

7. An implant delivery system for disrupting electrical signals traveling along a pulmonary vein by bidirectional stretching of the pulmonary vein wall, the system comprising:
a delivery catheter having a shaft with a distal end to a proximal end and lumen between the distal and proximal ends, the delivery catheter being configured for insertion into a patient's vascular system to position the distal end proximate a pulmonary vein location;
one or more implants having a radial expandable ring or coil configured to deploy within the pulmonary vein, the ring or coil being configured to deliver a force against pulmonary vein wall to provide bidirectional stretching of the pulmonary vein wall in both a radial direction and axial direction to stretch and create micro-tears in the pulmonary vein wall and then to hold open at a diameter slightly larger than normal without recoil; and
a delivery device having shaft with a distal end and proximal end, the one or more implants being positioned proximate the distal end of the delivery device;
wherein as the implant exit distally from the delivery catheter lumen, exposing the implant within the pulmonary vein for radial deployment.

8. The system of claim 7, wherein bidirectional stretching creates a two-step biological response in the pulmonary vein wall to promote cellular decoupling, comprising:
first, an acute response is caused by pressure-induced apoptosis inhibiting chemical exchange of sodium/calcium and disrupting focal electrical wave propagation; and
second, a biological response for chronic or long-term isolation/denervation is provided by causing focal endothelial cell proliferation at the implant site.

9. The system of claim 7, wherein the implant is self-expanding.

10. The system of claim 7, further comprising one or more balloons to expand the implant.

11. The system of claim 7, further comprising an imaging device.

12. The system of claim 7, further comprising an ablation device.

13. The system of claim 7, wherein the delivery device is configured to deliver one implant at a time, so that multiple implants may be implanted into a pulmonary vein, or individual implants may be implanted into different pulmonary vein during the same procedure.

14. The system of claim 7, wherein the delivery catheter is constructed of sufficiently flexible material to allow insertion through the tortuosity imposed by the patient's vascular system.

15. The system of claim 7, wherein the delivery device shaft being constructed of sufficiently flexible material to allow insertion through lumen of the delivery catheter, either during insertion of the delivery catheter or inserted through lumen after the delivery catheter is positioned within the pulmonary vein.

16. An implant device for disrupting electrical signals traveling along a pulmonary vein by bidirectional stretching, the implant device comprising:
a radial expandable ring or coil configured to deploy within the pulmonary vein and deliver a force against pulmonary vein wall to provide bidirectional stretching of the pulmonary vein wall in the radial direction and axial direction;
wherein bidirectional stretching creates a two-step biological response in the pulmonary vein wall to promote cellular decoupling, comprising:
an acute response is caused by pressure-induced apoptosis inhibiting chemical exchange of sodium/calcium and disrupting focal electrical wave propagation; and
second, a biological response for chronic or long-term isolation/denervation is provided by causing focal endothelial cell proliferation at the implant site.

17. An implant device for disrupting electrical signals traveling along a pulmonary vein by bidirectional stretching, the implant device comprising:
a radial expandable ring or coil configured to deploy within the pulmonary vein and deliver a force against pulmonary vein wall to provide bidirectional stretching of the pulmonary vein wall in the radial direction and axial direction;
wherein bidirectional stretching causes:
a first effect of stretching and micro-tears resulting in the creation of scar tissue formation, the scar tissue at least partially blocking and/or disrupting electrical conduction along the first pulmonary vein; and
a second effect of outward pressure causing the myocytes to be compressed, reducing their ability to perform normal function.

18. A method of treating an arrhythmia by bidirectional stretching, comprising:
selecting a patient;
choosing a first implant device for insertion into a first pulmonary vein of the patient;
inserting an implant device delivery catheter into the patient, wherein the implant delivery catheter comprises a distal end and a distal portion and the first implant device is positioned in the distal portion;
advancing the distal portion of the implant delivery device into the first pulmonary vein;
positioning the first implant device relative to the first pulmonary vein near the ostium and or left atrial—pulmonary vein junction;
deploying the first implant device within the first pulmonary vein near the ostium and or left atrial—pulmonary vein junction;
radially expanding to a diameter larger than the pulmonary vein, the first implant device to cause:
a first effect of stretching and micro-tears resulting in the creation of scar tissue formation, the scar tissue at least partially blocking and/or disrupting electrical conduction along the first pulmonary vein; and
a second effect of outward pressure causing the myocytes to be compressed, reducing their ability to perform normal function;
withdrawing the distal end of the implant delivery device from the first pulmonary vein.

19. The method of claim 1, wherein the first effect is selected from the group consisting of:
stretching of a portion of the pulmonary vein causing micro-tears in a portion of the pulmonary vein;
stretching of a portion of the pulmonary vein ostium causing micro-tears in a portion of the pulmonary vein ostium;
stretching of a portion of the left atrial—pulmonary vein junction causing micro-tears in a portion of the left atrial—pulmonary vein junction; and
combinations thereof.

20. The method of claim 1, wherein the second effect is selected from the group consisting of:
outwards pressure applied to the pulmonary vein;
outwards pressure applied to the pulmonary vein ostium; and
outwards pressure applied to the left atrial—pulmonary vein junction; and
combinations thereof.
